(19) United States
(12) Patent Application Publication
(10) Pub. No.: US 2010/0160742 A1
(43) Pub. Date: Jun. 24, 2010

(54) TELEMETRY SYSTEM AND METHOD

(75) Inventors: Neal John Seidl, Pewaukee, WI (US); Leo Zielinski, New Berlin, WI (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 12/338,512

(22) Filed: Dec. 18, 2008

(51) Int. Cl. A61B 5/0402; A61B 5/145

(52) U.S. Cl. 600/301; 600/481; 600/509

(57) ABSTRACT

An telemetry system is disclosed herein. The telemetry system includes a sensor configured to obtain cardiac data, a first wireless device configured to store identification data, and a transmitter connected to the sensor. The transmitter includes a second wireless device. The transmitter is configured to directly receive the cardiac data from the sensor, and to implement the second wireless device to receive the identification data from the first wireless device. The telemetry system also includes a receiver wirelessly coupled with the transmitter. The receiver is configured to receive the cardiac data and the identification data from the transmitter. The telemetry system also includes a processor coupled with the receiver. The processor is configured to correlate the cardiac data with the identification data.

### BACKGROUND OF THE INVENTION

This disclosure relates to a telemetry system and method. More specifically, this disclosure relates to a telemetry system and method adapted to monitor cardiac activity such as with an electrocardiogram (ECG).

An electrocardiograph is a device configured to record the electrical activity of the heart over time, and to convey the recorded electrical activity in the form of an ECG. The electrocardiograph operates by measuring electrical potential between various locations on the patient's body. The electrical potential measurements are obtained with a plurality of sensors secured directly to the patient. The sensors are operatively connected to a corresponding plurality of lead wires that are typically physically connected to a signal acquisition device. In a typical hospital environment, one or more electrocardiograph devices are implemented to obtain ECGs from each of a large number of different patients.

One problem is that the process of manually correlating each ECG with an appropriate patient is labor intensive. Another problem is that the process of manually correlating each ECG with an appropriate patient is subject to human error. These problems are compounded in the context of a telemetry system in which a large number of patients are being monitored.

### SUMMARY OF THE INVENTION

The above-mentioned shortcomings, disadvantages and problems are addressed herein which will be understood by reading and understanding the following specification.

In one embodiment, a telemetry system includes a sensor configured to obtain cardiac data, a first wireless device configured to store identification data, and a transmitter connected to the sensor. The transmitter includes a second wireless device. The transmitter is configured to directly receive the cardiac data from the sensor, and to implement the second wireless device to receive the identification data from the first wireless device. The telemetry system also includes a receiver wirelessly coupled with the transmitter. The receiver is configured to receive the cardiac data and the identification data from the transmitter. The telemetry system also includes a processor coupled with the receiver. The processor is configured to correlate the cardiac data with the identification data.

In another embodiment, a method includes storing identification data on a wireless device, implementing a sensor to obtain cardiac data, detecting and acquiring the identification data and the cardiac data, and implementing a processor to correlate the cardiac data with a specific patient based on the identification data.

Various other features, objects, and advantages of the invention will be made apparent to those skilled in the art from the accompanying drawings and detailed description thereof.

### BRIEF DESCRIPTION OF THE DRAWINGS

FIG. 1 is a schematic representation of a telemetry system in accordance with an embodiment;

FIG. 2 is a schematic representation of the telemetry system of FIG. 1 in accordance with another embodiment; and

FIG. 3 is flow chart illustrating a method in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

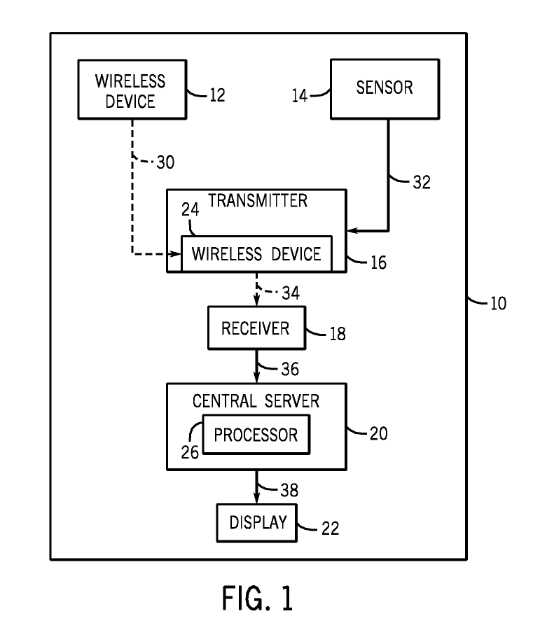

Referring to FIG. 1, a telemetry system 10 is shown in accordance with an embodiment. The telemetry system 10 comprises a first wireless device 12; a sensor 14; a transmitter 16; a receiver 18; a central server 20; and a display 22. The transmitter 16 comprises a second wireless device 24 that is adapted to function in a complementary manner with the first wireless device 12 to transfer data. The central server 20 comprises a processor 26.

The first wireless device 12 will hereinafter be referred to as an RFID transponder 12, and the second wireless device 24 will be referred to as an RFID reader 24 in accordance with an embodiment. It should, however, be appreciated that other wireless devices may be envisioned such as, for example, a bar code and a bar code reader. The RFID transponder 12 is wirelessly coupled with the RFID reader 24 of the transmitter 16 by the wireless connection 30 that is represented by a dashed line. The RFID transponder 12 may be configured to transmit data to the RFID reader 24 of the transmitter 16. The RFID transponder 12 may be referred to as being wirelessly detectable, in the sense that it can be detected by the RFID reader 24 without requiring that a fixed-wire connection be established between the RFID transponder 12 and the RFID reader 24. In other words, the RFID reader 24 can detect the RFID transponder 12 when they are in sufficiently close proximity, and subsequently the RFID reader 24 can acquire data from the RFID transponder 12.

The RFID transponder 12 is configured to retain identification data and to transmit the identification data to the RFID reader 24 of the transmitter 16. The identification data may, for example, comprise the patient's identity as well as data pertaining to the patient's, age, height, weight, sex, race, family and genetic medical data, medical history, physical handicaps, known medical conditions, known medical allergies, and current ailment conditions such as symptoms, duration, physician observations and the like. As another example, the identification data retained may comprise an arbitrary unique ID that has separately been associated with the patient's identity and/or other identification data, retained outside of the RFID transponder.

In a non-limiting manner, the RFID transponder 12 may be may be embedded in an adhesive tag (not shown) that adheres to the patient in a tamper resistant fashion, or disposed within a wristband (not shown) adapted for attachment to a patient's wrist. During the admissions process, a patient's identification data may be manually input and stored on the RFID transponder 12. The RFID transponder 12 can then be secured directly to the patient such that the identification data is physically associated with the appropriate patient.

The sensor 14 is connectable to the transmitter 16 via connection 32 that is represented by a solid line. Connection 32 generally comprises a wire or other conductor adapted to electrically couple the sensor 14 with the transmitter 16. According to one embodiment, the sensor 14 comprises a plurality of electrocardiogram (ECG) lead electrodes, and the connection 32 comprises a corresponding plurality of ECG lead wires. According to another embodiment, the sensor 14 comprises a pulse oximetry device adapted for attachment to a patient's finger.

The sensor 14 monitors cardiac activity of a patient. More precisely, the sensor 14 is configured to obtain cardiac data from a patient, and to transmit the cardiac data to the transmitter 16 via connection 32. According to the embodiment wherein the sensor 14 comprises a plurality of ECG lead electrodes, the cardiac data is convertible into ECG data comprising a P-wave, a QRS complex and a T-wave and/or heart rate information in a known manner.

The transmitter 16 is adapted to receive identification data from the RFID transponder 12 via connection 30, and cardiac data from the sensor 14 via connection 32. The transmitter 16 is further adapted to transmit the identification data and the cardiac data to the receiver 18 via wireless connection 34 represented by a dashed line. According to one embodiment, the transmitter 16 is a compact device that may be conveniently carried by a patient.

The receiver 18 is adapted to automatically detect and receive data from the transmitter 16 via connection 34. The receiver 18 is further configured to transmit the identification data and cardiac data to the central server 20 via connection 36. Connection 36 generally comprises a wire or other conductor adapted to electrically couple the receiver 18 with the central server 20. Transmitters and receivers are well known in the art and thus will not be explained in further detail.

The central server 20 is adapted to receive identification data and cardiac data from the receiver 18, and to selectively transmit data to the display 22. The central server 20 comprises a processor 26 adapted to automatically correlate the cardiac data with a specific patient based on the identification data. As this process was conventionally manually performed, the implementation of the central server 20 in the manner described reduces labor requirements and human error. According to one embodiment, the processor 26 converts the cardiac data into ECG data comprising a P-wave, a QRS complex and a T-wave, labels the ECG data with the associated patient's identity, and transmits the labeled ECG data to the display 22 via connection 38.

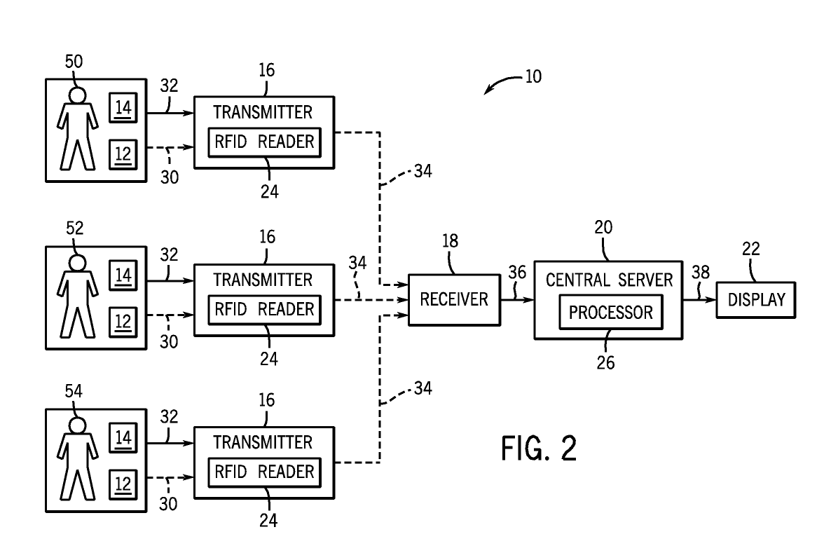

Having described the individual components of the telemetry system 10 in detail, the telemetry system 10 will now be described in accordance with a non-limiting, exemplary embodiment shown in FIG. 2. Common reference numbers are implemented to identify similar components in FIGS. 1 and 2. For purposes of this exemplary embodiment, assume that three patients 50-54 are being monitored by the telemetry system 10. It should, however, be appreciated that the telemetry system 10 may be implemented to monitor a much larger number of patients.

The patents 50-54 each have an RFID transponder 12 and a sensor 14. Assume for purposes of this embodiment that a separate RFID transponder 12 is secured to each of the patients 50-54 with a wristband, and that the sensor 14 attached to each patient 50-54 comprises a plurality of ECG lead electrodes. It should also be assumed that the RFID transponder 12 associated with each patient 50-54 has been pre-programmed with the patient's identification data.

For purposes of the present embodiment, the transmitter 16 comprises a compact pocket-sized device that may be conveniently carried by the patient. By carrying the transmitter 16 wherever they go, the patient can be generally continuously monitored from a variety of different locations within or near a given hospital facility. In contrast, more conventional systems are only capable of monitoring patients while they are in bed. Also for purposes of the present embodiment, the receiver 18 comprises a plurality of receiver devices disposed throughout a hospital facility such that a network is formed and each transmitter 16 is detectable from a plurality of different locations.

Cardiac data from each patient 50-54 can be acquired with a sensor 14, and transmitted to the central server 20 in the manner previously described. Advantageously, this cardiac data acquisition can take place on multiple patients and from a plurality of different locations within a hospital facility. One problem with conventional systems, particularly those adapted to monitor much larger numbers of patients, it is that it is necessary to ensure a given set of cardiac data is associated with the appropriate patient. By implementing the telemetry system 10 to automatically correlate cardiac data with a specific patient based on the identification data from the RFID transponder 12, labor requirements and the potential for human error are minimized.

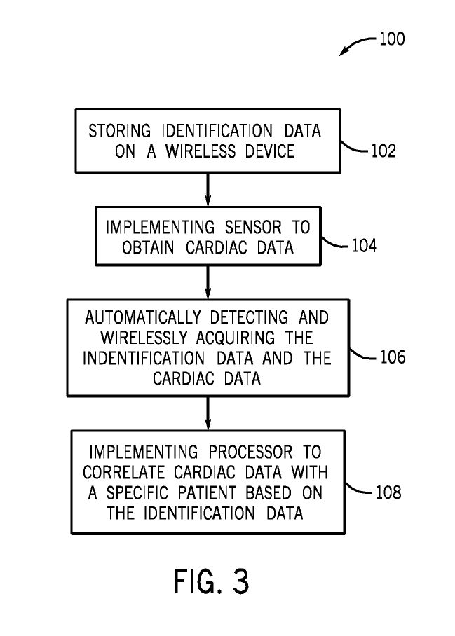

Referring to FIG. 3, a method 100 for implementing the telemetry system 10 will now be described in accordance with an embodiment. The method 100 comprises a plurality of steps 102-110. Steps 102-110 need not necessarily be performed in the order shown.

At step 102, patient identification data is stored on a wireless device such as, for example, an RFID transponder. This step is typically manually performed when the patient is admitted to a hospital. Step 102 may also optionally comprise securing the RFID transponder to a corresponding patient with a wristband. At step 104, a sensor is implemented to obtain cardiac data. The sensor may, for example, comprise a plurality of ECG lead electrodes.

At step 106, the identification data and cardiac data are detected and wirelessly acquired. This step is preferably performed by the transmitter 16 and receiver 18 (shown in FIG. 1). More precisely, the RFID reader 24 of the transmitter 16 detects and wirelessly acquires the identification data from the RFID transponder 12. Similarly, the receiver 18 detects and wirelessly acquires both the identification data and the cardiac data from the transmitter 16.

At step 108, the processor 26 (shown in FIG. 1) is implemented to correlate the cardiac data with a specific patient based on the identification data. This step may also optionally include converting the cardiac data into ECG data comprising a P-wave, a QRS complex and a T-wave, and labeling the ECG data with the associated patient's identity.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A telemetry system comprising:
a sensor configured to obtain cardiac data;
a first wireless device configured to store identification data;
a transmitter connected to the sensor, said transmitter comprising a second wireless device, said transmitter configured to directly receive the cardiac data from the sensor, and to implement the second wireless device to receive the identification data from the first wireless device;
a receiver wirelessly coupled with the transmitter, said receiver configured to receive the cardiac data and the identification data from the transmitter; and
a processor coupled with the receiver, said processor configured to correlate the cardiac data with the identification data.

2. The telemetry system of claim 1, wherein the sensor comprises a plurality of electrocardiogram lead electrodes.

3. The telemetry system of claim 1, wherein the sensor comprises a pulse oximeter.

4. The telemetry system of claim 1, wherein the first wireless device comprises an RFID transponder and the second wireless device comprises an RFID reader.

5. The telemetry system of claim 4, further comprising a wristband adapted to retain the RFID transponder.

6. The telemetry system of claim 1, wherein the first wireless device comprises a bar code and the second wireless device comprises a bar code reader.

7. The telemetry system of claim 1, wherein the processor is configured to convert the cardiac data to electrocardiogram data.

8. The telemetry system of claim 1, further comprising a display in communication with the processor, said display configured to visually convey the cardiac data.

9. (canceled)

10. (canceled)

11. (canceled)

12. (canceled)

13. (canceled)

14. (canceled)

15. (canceled)
